SEQUENCE LISTING (6 sequences)

SEQ ID NO: 1 - DNA, 40 bp, Human papilloma virus
atatgtgcttctacacagtctcctgtacctgggcaatatg

SEQ ID NO: 2 - DNA, 23 bp, Artificial Sequence
  Description of Artificial Sequence Synthetic oligonucleotide
atttaggtgacactatagaagcg

SEQ ID NO: 3 - DNA, 86 bp, Artificial Sequence
  Description of Artificial Sequence Synthetic probe
taatacgactcactatagggagacatattgcccaggtacaggagactgtgtagaagcacatatcgcttctatagtgtcacctaaat

SEQ ID NO: 4 - DNA, 86 bp, Artificial Sequence
  Description of Artificial Sequence Synthetic oligonucleotide
atttaggtgacactatagaagcgatatgtgcttctacacagtctcctgtacctgggcaatatgtctccctatagtgagtcgtatta

SEQ ID NO: 5 - RNA, 69 nt, Artificial Sequence
  Description of Artificial Sequence Synthetic oligonucleotide
gggagacauauugcccagguacaggagacuguguagaagcacauaucgcuucuauagugucaccuaaau

SEQ ID NO: 6 - RNA, 69 nt, Artificial Sequence
  Description of Artificial Sequence Synthetic oligonucleotide
gaagcgauaugugcuucuacacagucuccuguaccugggcaauaugucucccuauagugagucguauua

(19) United States
(12) Patent Application Publication
(10) Pub. No.: US 2009/0181389 A1
(43) Pub. Date: Jul. 16, 2009

(54) QUANTITATIVE MEASUREMENT OF NUCLEIC ACID VIA LIGATION-BASED LINEAR AMPLIFICATION

(75) Inventors: Xianqiang Li, Palo Alto, CA (US); Xin Jiang, Saratoga, CA (US)

Correspondence Address: WILSON SONSINI GOODRICH & ROSATI, 650 PAGE MILL ROAD, PALO ALTO, CA, 94304-1050, US

(73) Assignee: Signosis, Inc., a California Corporation

(21) Appl. No.: 12/263,099

(22) Filed: Oct. 31, 2008

Related U.S. Application Data
(60) Provisional application No. 61/020,692, filed on Jan. 11, 2008.

(51) Int. Cl. C12Q 1/68

(52) U.S. Cl. 435/6

(57) ABSTRACT

Methods, compositions and kits are provided for sensitive and quantitative detection of nucleic acid, especially for the determination of the presence and/or amount of a target nucleic acid with mutations or single nucleotide polymorphism (SNP). In particular, assays are provided for amplifying a target nucleic acid via ligation of designed oligonucleotide probes and linear amplification by using an RNA polymerase, such as T7 polymerase. The assays can be used for diagnosis, prognosis or monitoring of diseases or disorders, for pharmacogenomic studies of patient stratification and drug responses, for discovery of therapeutic targets, or for forensic analysis.

### CROSS-REFERENCE

This application claims the benefit of U.S. Provisional Application No. 61/020,692, filed Jan. 11, 2008, which application is incorporated herein by reference in its entirety.

### BACKGROUND OF THE INVENTION

Nucleic acid analysis is becoming an important tool for the diagnosis and prognosis of infectious as well as genetic diseases. The inheritance of a substantial number of disease traits can be predicted by genetic analysis. For instance, new discovered microRNAs (miRNAs) are important to the regulation of gene expression. These small molecules inhibit protein production through selective binding to the complementary messenger RNA sequences. Although the inhibition-mediated biological function of these miRNA molecules are not yet fully understood, miRNAs seems to be crucial in diverse regulations, including development, cell differentiation, proliferation, apoptosis, and maintenance of stemness and imprinting. Moreover, for an increasing number of genetic diseases, the genes involved have been identified and mutant alleles characterized.

Large-scale multiplex analysis of nucleic acid is needed for practical identification of individuals, e.g., for paternity testing and in forensic science, for organ-transplant donor-recipient matching, for genetic disease diagnosis, prognosis, and pre-natal counseling, and the study of oncogenic mutations. In addition, the cost-effectiveness of infectious disease diagnosis by nucleic acid analysis varies directly with the multiplex scale in panel testing. Many of these applications depend on the discrimination of single-base differences at a multiplicity of sometimes closely spaced loci.

Although there are many techniques currently used to detect target nucleic acids, the need remains for a rapid single assay format to detect the presence or absence of multiple selected sequences in a polynucleotide sample.

### SUMMARY OF THE INVENTION

The invention relates to methods, compositions and devices, e.g., for detecting a target nucleic acid in a sample.

In one aspect, the invention provides a method for detecting a target nucleic acid in a sample. In some embodiments of this aspect, the invention provides an oligonucleotide probe set. In some embodiments, the invention provides at least one oligonucleotide probe set, each set containing a first oligonucleotide probe having a 5′ target specific region and a 3′ universal sequence region, and a second probe having a 3′ target specific region and a 5′ phage promoter region, where the first and the second oligonucleotide probes are suitable for ligation together when hybridized adjacent to one another to the target nucleic acid. The oligonucleotide probe set is annealed to the target nucleic acid such that a complex is formed between the target nucleic acid and the oligonucleotide probes. The complex is then contacted with a linking agent such that the directly adjacent 5′ and 3′ ends of the first and second probes covalently bond to form a ligated probe product. A primer is annealed to the 3′ universal sequence region of the first oligonucleotide probe in the ligated probe product and contacted with a polymerase under conditions such that the annealed primer is extended to form extension products complementary to the sequences to which the primers is annealed to form a double stranded nucleic acid product. In some embodiments, the 5′ phage promoter region of the second oligonucleotide probe in the double stranded nucleic acid product is contacted with a phage polymerase under conditions such that a transcription product of the phage promoter region is formed; and the presence of the transcription product is detected, where the presence of the transcription product is indicative of the presence of the target nucleic acid in the sample. In some embodiments, the first and second oligonucleotide probes have a predetermined sequence.

In some embodiments, the phage promoter region of the second oligonucleotide probe is selected from the group consisting of T7 RNA polymerase promoter, T3 RNA polymerase promoter or SP6 RNA polymerase promoter. In some embodiments, the universal sequence region of the first oligonucleotide probe is SP6 RNA polymerase promoter.

In some embodiments, the transcription product is detected using a DNA microarray, bead microarray, high throughput sequencing, or single microtiter plate assay. In some embodiments, the transcription product is detected using branched DNA. In some embodiments, the transcription product has a detectable label. The detectable label can be a fluorescent or biotin label, and the step of detecting includes detecting a fluorescent signal generated by the fluorescent or chemiluminescent or color. In some embodiments, the label is incorporated during the transcription of the phage promoter region of the second oligonucleotide probe. In some embodiments, the incorporation of label includes adding a label nucleotide to the transcription of the phage promoter region of the second oligonucleotide probe.

In some embodiments, the target nucleic acid is DNA or RNA. In some embodiments, the DNA or RNA is derived from genomic DNA or total RNA.

In some embodiments, the method further comprises separating the complex from the non-annealed first and second oligonucleotide probes. In some embodiments, the first oligonucleotide further comprises a capturing portion. The capturing portion can be used to separate the annealed complex from the non-annealed first and second oligonucleotide probes. The capturing portion can be used to separate the ligated probe product from unligated first and second oligonucleotide probes. Examples of capturing portions include, but are not limited to, biotin and a capture sequence. In some embodiments, the capturing portion is biotin. In some embodiments, the ligated probe product is isolated by binding said biotin with a strepavidin bound to a solid support.

In some embodiments, the primer annealed to the universal sequence of said first oligonucleotide further comprises a capturing portion. The capturing portion can be used to separate the ligated probe product from unligated first and second oligonucleotide probes. Examples of capturing portions include, but are not limited to, biotin and a capture sequence. In some embodiments, the capturing portion is biotin. In some embodiments, the ligated probe product is isolated by binding biotin with a strepavidin bound to a solid support.

In some embodiments, the first oligonucleotide probe contains in 5′ to 3′ order a target specific region, a tag region and a phage promoter region. In some embodiments, the second oligonucleotide probe contains in 3′ to 5′ order a target specific region, a tag region and a phage promoter region.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 schematically illustrates an embodiment of the invention of target nucleic acid annealing with two stacking oligos to form a target nucleic acid/DNA hybrid.

FIG. 2 schematically illustrates an embodiment of the invention of target nucleic acid annealing with three stacking oligos to form a target nucleic acid/DNA hybrid.

FIG. 3: illustrates an overview for a T7-OLA/PCR assay.

FIG. 4: illustrates an overview for a phage promoter-OLA/PCR assay to detect an HPV18 suptype.

FIG. 5: illustrates an overview for a phage promoter-OLA/PCR assay to detect an HPV18 suptype.

FIG. 6 schematically illustrates an embodiment of the invention for bDNA detection in a T7 transcribed RNA array assay.

FIG. 7 schematically illustrates an embodiment of the invention for bDNA detection.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entirety.

The assay of the present invention is particularly useful for analyzing nucleic acids (DNA or RNA). The methods described herein provide a sensitive assay for determining the presence or absence of a target nucleic acid, e.g., the presence of absence of a point mutation or a SNP in a target nucleic acid. In some embodiments, the method described herein use oligonucleotide probes which are complementary to two contiguous predetermined sequences of the test substance. If these probes anneal in a juxtaposed position, there is a reasonable certainty that the sequence being investigated is the relevant one. The annealed probes are then exposed to a linking agent which then ligates the adjacent ends of the probes if the nucleotides base pair at the target nucleotide position. Then, the presence or absence of ligation is determined by one of a number of techniques to be described below.

The oligonucleotide probe sets can be in the form of any nucleotide such as ribonucleotides, deoxynucleotides, modified ribonucleotides, modified deoxyribonucleotides, peptide nucleotide analogues, modified peptide nucleotide analogues, modified phosphate-sugar-backbone oligonucleotides, nucleotide analogs, and mixtures thereof. In some embodiments, the oligonucleotide probe sets are in the form of deoxynucleotides.

The linking agent could be a ligase. In some embodiments the ligase is T4 DNA ligase, using well known procedures (Maniatis, T. in Molecular Cloning, Cold Spring Harbor Laboratory (1982)). Other DNA ligases may also be used. T4 DNA ligase may also be used when the target nucleic acid is RNA (The Enzymes, Vol. 15 (1982) by Engler M. J. and Richardson C. C., p. 16-17. Methods in Enzymology, Vol. 68 (1979) Higgins N. P. and Cozzarelli N. R. p. 54-56). With regard to ligation, other ligases, such as those derived from thermophilic organisms may be used thus permitting ligation at higher temperatures allowing the use of longer probes (with increased specificity) which could be annealed and ligated simultaneously under the higher temperatures normally associated with annealing such probes. The ligation, however, need not be by an enzyme and, accordingly, the linking agent may be a chemical agent which will cause the probes to link unless there is a nucleotide base pair mismatching at the target nucleotide position. For simplicity, some embodiments of the invention will be described using T4 DNA ligase as the linking agent. This enzyme requires the presence of a phosphate group on the 5′ end that is to be joined to a 3′ OH group on a neighboring oligonucleotide.

In some cases, the methods described herein involve performing one or more genetic analyses or detection steps on nucleic acids. In some embodiments target nucleic acids are from a sample obtained from an animal. Such animal can be a human or a domesticated animal such as a cow, chicken, pig, horse, rabbit, dog, cat, or goat. Samples derived from an animal, e.g., human, can include, for example whole blood, sweat, tears, ear flow, sputum, lymph, bone marrow suspension, lymph, urine, saliva, semen, vaginal flow, cerebrospinal fluid, brain fluid, ascites, milk, secretions of the respiratory, intestinal or genitourinary tracts fluid. In some embodiments the sample is a cell sample. Cell samples can be obtained from a variety of tissues depending on the age and condition of the animal. Cell samples can be obtained from peripheral blood using well known techniques. In fetal testing, a sample can be obtained by amniocentesis, chorionic villi sampling or by isolating fetal cells from the blood of a pregnant individual. Other sources of nucleic acids include blood, semen, buccal cells, or the like. Nucleic acids can be obtained from any tissue or organ by methods well known in the art.

To obtain a blood sample, any technique known in the art may be used, e.g. a syringe or other vacuum suction device. A blood sample can be optionally pre-treated or processed prior to enrichment. Examples of pre-treatment steps include the addition of a reagent such as a stabilizer, a preservative, a fixant, a lysing reagent, a diluent, an anti-apoptotic reagent, an anti-coagulation reagent, an anti-thrombotic reagent, magnetic property regulating reagent, a buffering reagent, an osmolality regulating reagent, a pH regulating reagent, and/or a cross-linking reagent.

When a blood sample is obtained, a preservative such an anti-coagulation agent and/or a stabilizer can be added to the sample prior to enrichment. This allows for extended time for analysis/detection. Thus, a sample, such as a blood sample, can be analyzed under any of the methods and systems herein within 1 week, 6 days, 5 days, 4 days, 3 days, 2 days, 1 day, 12 hrs, 6 hrs, 3 hrs, 2 hrs, or 1 hr from the time the sample is obtained.

In some embodiments, a blood sample can be combined with an agent that selectively lyses one or more cells or components in a blood sample. For example, fetal cells can be selectively lysed releasing their nuclei when a blood sample including fetal cells is combined with deionized water. Such selective lysis allows for the subsequent enrichment of fetal nuclei using, e.g., size or affinity based separation. In another example platelets and/or enucleated red blood cells are selectively lysed to generate a sample enriched in nucleated cells, such as fetal nucleated red blood cells (fnRBC) and maternal nucleated blood cells (mnBC). The fnRBC's can subsequently be separated from the mnBC's using, e.g., affinity to antigen-i or magnetism differences in fetal and adult hemoglobin.

When obtaining a sample from an animal (e.g., blood sample), the amount can vary depending upon animal size, its gestation period, and the condition being screened. In some embodiments, up to 50, 40, 30, 20, 10, 9, 8, 7, 6, 5, 4, 3, 2, or 1 mL of a sample is obtained. In some embodiments, 1-50, 2-40, 3-30, or 4-20 mL of sample is obtained. In some embodiments, more than 5, 10, 15, 20, 25, 30, 35, 40, 45, 50, 55, 60, 65, 70, 75, 80, 85, 90, 95 or 100 mL of a sample is obtained.

Nucleic acids from samples that can be analyzed by the methods herein include: double-stranded DNA, single-stranded DNA, single-stranded DNA hairpins, DNA/RNA hybrids, RNA (e.g. mRNA or miRNA) and RNA hairpins. Examples of genetic analyses that can be performed on nucleic acids include e-g., SNP detection, STR detection, RNA expression analysis, promoter methylation, gene expression, virus detection, viral subtyping and drug resistance.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

In some embodiments, the methods described herein are used to detect and/or quantified a target nucleic acid molecule. In some embodiments, the methods described herein are used to detect and/or quantified multiple target nucleic acid molecules. The methods described herein can analyzed at least 1, 2, 3, 4, 5, 10, 20, 50, 100, 200, 500, 1,000, 2,000, 5,000, 10,000, 20,000, 50,000, 100,000, different target nucleic acids.

In some embodiments, the methods described herein are used to detect and/or quantify target nucleic acids to profile a specific tissue or a specific condition. In some embodiments, the methods described herein are used to detect and/or quantify target nucleic acids to detect biomarkers for specific tissue or condition. In some embodiments, the methods described herein are used to regulate gene expression. In some embodiments, the methods described herein are use for gene therapy. In some embodiments, the methods described herein are used to detect and/or quantify target nucleic acids to profile a neoplastic and/or cancer cell. In some embodiments, the methods described herein are used to detect and/or quantify target nucleic acids to diagnose cancer and/or a neoplastic condition. In some embodiments, the methods described herein are used to detect and/or quantify target nucleic acids to detect biomarkers in a neoplastic and/or cancer cell. In some embodiments, the methods described herein are used to regulate gene expression in a neoplastic and/or cancer cell. In some embodiments, the methods described herein are used for gene expression.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

In some embodiments, the methods described herein are used to distinguish between target nucleic acids that differ from another nucleic acid by 1 nt. In some embodiments, the methods described herein are used to distinguish between target nucleic acids that differ from another nucleic acid by 1 nt or more than 1, 2, 3, 5, 10, 15, 20, 21, 22, 24, 25, 30 nt.

In some embodiments, the methods described herein are used to quantify nucleic acid expression in different tissues, developmental lineages and/or different states of a condition. In some embodiments, the methods described herein are used to quantify nucleic acid expression in different states of a neoplastic and/or cancer condition.

In some embodiments, the methods described herein are used to detect and/or quantify target nucleic acids without the need of target nucleic acid isolation. In some embodiments, the methods described herein are used to detect and/or quantify a target nucleic acid directly from a nucleic acid sample comprising DNA and RNA molecules.

In some embodiments, the methods described herein are used to detect and/or quantify genomic DNA regions. In some embodiments, the methods described herein are used to diagnose a fetal abnormality. Aneuploidy means the condition of having less than or more than the normal diploid number of chromosomes. In other words, it is any deviation from euploidy. Aneuploidy includes conditions such as monosomy (the presence of only one chromosome of a pair in a cell's nucleus), trisomy (having three chromosomes of a particular type in a cell's nucleus), tetrasomy (having four chromosomes of a particular type in a cell's nucleus), pentasomy (having five chromosomes of a particular type in a cell's nucleus), triploidy (having three of every chromosome in a cell's nucleus), and tetraploidy (having four of every chromosome in a cell's nucleus). Birth of a live triploid is extraordinarily rare and such individuals are quite abnormal, however triploidy occurs in about 2-3% of all human pregnancies and appears to be a factor in about 15% of all miscarriages. Tetraploidy occurs in approximately 8% of all miscarriages. (http://www.emedicine.com/med/topic3241.htm).

In some embodiments, the methods described herein are used to detect and/or quantify genomic DNA regions to diagnose a fetal condition such as aneuploidy. In some embodiments, the methods described herein are used to diagnose a fetal abnormality by quantifying a DNA region chosen on a chromosome suspected of aneuploidy and on a control chromosome. In some embodiment aneuploidy is trisomy selected from the group consisting of: trisorny 13, trisomy 18, trisomy21 (Down Syndrome), Klinefelter Syndrome (X X Y), or other irregular number of sex or autosomal chromosomes, and a combination thereof. Examples of chromosomes that are often trisomic include chromosomes 21, 18, 13, and X. In some cases, 1 or more than 1, 2, 3, 4, 5, 6, 7, 8, 9, 10, 15, or 20 regions are detected and quantified per chromosome tested. In some embodiments, the methods described herein can discriminate and quantitate genomic DNA regions. The methods described herein can discriminate and quantitate genomic DNA regions of at least 1, 2, 3, 4, 5, 10, 20, 50, 100, 200, 500, 1,000, 2,000, 5,000, 10,000, 20,000, 50,000, 100,000, different genomic DNA regions. The methods described herein can discriminate and quantitate genomic DNA regions varying by 1 nt or more than 1, 2, 3, 5, 10, 15, 20, 21, 22, 24, 25, 30 nt.

In some embodiments, the methods described herein are used to detect and/or quantify genomic DNA regions such as a region containing a DNA polymorphism. A polymorphism refers to the occurrence of two or more genetically determined alternative sequences or alleles in a population. A polymorphic marker or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include single nucleotide polymorphisms (SNP's), restriction fragment length polymorphisms (RFLP's), variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens. In some embodiments, the methods described herein can discriminate and quantitate a DNA region containing a DNA polymorphisms. The methods described herein can discriminate and quantitate DNA polymorphism of at least 1, 2, 3, 4, 5, 10, 20, 50, 100, 200, 500, 1,000, 2,000, 5,000, 10,000, 20,000, 50,000, 100,000, different genomic DNA regions.

In some embodiments, the methods described herein are used to detect and/or quantitate a DNA epigenetic change. In some embodiments, the DNA epigenetic change is selected for the group consisting of chemical modifications and chromatin structure. In some embodiments the DNA epigenetic change is a chemical modification. In some embodiments, the chemical modification is DNA methylation.

The present invention provides a method for determining methylation status of CpG dinucleotides within a target nucleic acid molecule. CpG islands (a stretch of CpGs), are typically unmethylated. Hypermethylation in CpG islands of promoter regions leads to silence the associated gene expression. Aberrant methylation has been associated to different pathogenesis including neoplasia. In some embodiments, the methods described herein can discriminate and quantitate the methylation state of at least 1, 2, 3, 4, 5, 10, 20, 50, 100, 200, 500, 1,000, 2,000, 5,000, 10,000, 20,000, 50,000, 100,000, different target nucleic acids. In some embodiments, the methods described herein are used to detect and/or quantify methylation status of target nucleic acids with similar sequences. The methods described herein can discriminate and quantitate the methylation state of target nucleic acids varying by 1 nt or more than 1, 2, 3, 4, 5, 10, 12, 15, 20 nt.

In some embodiments, the methods described herein are used to detect and/or quantify gene expression. In some embodiments, the methods described herein provide high discriminative and quantitative analysis of multiples genes. The methods described herein can discriminate and quantitate the expression of at least 1, 2, 3, 4, 5, 10, 20, 50, 100, 200, 500, 1,000, 2,000, 5,000, 10,000, 20,000, 50,000, 100,000, different target nucleic acids.

In some embodiments, the methods described herein are used to detect and/or quantify gene expression of genes with similar sequences. The methods described herein can discriminate and quantitate the expression of genes varying by 1 nt or more than 1, 2, 3, 4, 5, 10, 12, 15, 20, 21, 22, 24, 25, 30 nt.

For instance, CYP encodes P450 proteins that play important roles in the metabolism of xenobiotic chemicals. The human genome encodes fifty seven P450 proteins. The expression of some CYPs is highly regulated. Profiling of CYP gene expression is difficult as sequence similarity of these genes. They are hardly differentiated by direct hybridization based on their own sequences or their not quantitatively analyzed due to non-linear amplification. In some embodiments, the methods described herein can discriminate and quantitate the expression of CYP genes. The methods described herein can discriminate and quantitate the expression of at least 1, 2, 3, 4, 5, 10, 20, 50, 57, different CYP genes. The methods described herein can discriminate and quantitate the expression of CYP genes varying by 1 nt or more than 1, 2, 3, 4, 5, 10, 12, 15, 20 nt.

In some embodiments, the methods described herein are used to detect and/or quantify microRNAs (miRNAs). New discovered miRNAs are thought to be important in the regulation of gene expression. MiRNA are usually single-stranded RNAs approximately 22 nt long. Without being limited to any theory, these small molecules inhibit protein production through selective binding to the complementary messenger RNA sequences. Although the inhibition-mediated biological function of these miRNA molecules are not yet fully understood, miRNAs seems to be crucial in diverse regulations, including development, cell differentiation, proliferation, apoptosis, and maintenance of stemness and imprinting. Through selective binding to complementary messenger RNA sequences, they can mediate translation repression or RNA degradation. Up to 20%-25% of mammalian genes might be regulated by miRNAs. So far, more than 400 miRNAs have been identified in human genome. Many of them are only different in one or few nucleotides (http://microrna.sanger.ac.uk/sequences/ftp.shtml).

In some embodiments, the methods described herein are used to detect and/or quantified a miRNA molecule. In some embodiments, the methods described herein are used to detect and/or quantified multiple miRNA molecules. The methods described herein can analyzed at least 1, 2, 3, 4, 5, 10, 20, 50, 100, 200, 500, 1,000, 2,000, 5,000, 10,000, 20,000, 50,000, 100,000, different miRNAs.

Recent studies have shown that expression of mature miRNAs is tissue-specific and the abundance of miRNAs varies several orders of magnitude. In some cases, expression of miRNAs is tissue-specific, such as the expression of miRNAs miR-1 and miR-133 to be specific to heart and skeletal muscle and miR-122a specific to liver tissue. Moreover, miss-regulation of miRNA expression might contribute to human cancers and miRNAs are considered to be a new class of cellular molecules involved in human oncogenesis. miRNA has been demonstrated to be a new class of cellular molecules involved in human oncogenesis. The first report was made in chronic lymphatic leukemia (CLL) where a number of patients show down-regulation of miRNA-15 and miRNA-16. These studies were followed by studies demonstrating altered expression of miRNA in a number of cancers including colon cancers, Burkitt lymphoma, lung cancer, breast cancer, large cell lymphoma, glioblastoma, B cell lymphoma, hepatocellular carcinoma, and papillary thyroid carcinoma. Expression of mature miRNA is also found to be specific to normal but not cancer cells and tissues. For instance, the expression of mature miR-122a is very low in four liver cancer cell lines and hepatocellular carcinomas, but very high in normal liver tissue. Systemically profiling of miRNA expression displays unique signatures in a number of cancers, such as the difference that can differentiate malignant and non-malignant prostate samples, and discriminate clinically relevant breast cancer phenotypes.

In some embodiments, the methods described herein are used to detect and/or quantify miRNAs to profile a specific tissue or a specific condition. In some embodiments, the methods described herein are used to detect and/or quantify miRNAs to detect biomarkers for specific tissue or condition. In some embodiments, the methods described herein are used to regulate gene expression. In some embodiments, the methods described herein are use for gene therapy. In some embodiments, the methods described herein are used to detect and/or quantify miRNAs to profile a neoplastic and/or cancer cell. In some embodiments, the methods described herein are used to detect and/or quantify miRNAs to diagnose cancer and/or a neoplastic condition. In some embodiments, the methods described herein are used to detect and/or quantify miRNAs to detect biomarkers in a neoplastic and/or cancer cell. In some embodiments, the methods described herein are used to regulate gene expression in a neoplastic and/or cancer cell. In some embodiments, the methods described herein are used for gene expression.

miRNAs are found in the genomes of humans, animals, plants and viruses. miRNAs are generated from endogenous hairpin-shaped transcripts. In animals, miRNAs are transcribed as long primary transcripts (pri-microRNAs) by RNA polymerase II enzyme. They are cleaved in the nucleus by RNAse III endonuclease Drosha, releasing a ˜60-70 nt stem loop pre-miRNAs. The pre-miRNA is actively transported to the cytoplasm by export receptor exportin-5 where it is processed by the enzyme Dicer yielding a ˜22 nt microRNA duplexes. Following denaturation by the action of helicases, one strand of the duplex (the mature miRNA) is incorporated into a ribonucleoprotein complex known as RISC (RNA-induced silencing complex), which will guide the particular miRNA to its messenger RNA target to lead to regulation of the corresponding protein. In some embodiments, the methods described herein are used to distinguish precursor miRNAs from mature miRNA. The methods described herein can distinguish at least 1, 2, 3, 4, 5, 10, 20, 50, 100, 200, 500, 1,000, 2,000, 5,000, 10,000, 20,000, 50,000, 100,000, different miRNAs from their precursor.

Many miRNA have been identified through both biological approach and informatics analysis. There are total 475 human miRNA genes listed in the miRNA database (http://microma.sanger.ac.uk/sequences/ftp.shtml) and it is expected to be approximately 1000, which would be equivalent to almost 3% of the protein-coding genes. Many of mature human miRNAs are closely related in sequences and more than 20% are grouped into isoforms with nearly identical sequences, usually differing by 1-3 nt. The largest human isoform families include let-7, including 9 mature molecules with different sequences. These families are designated with a letter (e.g. let-7b and let-7c). Distinguished miRNA expression has found in the different tissues, developmental lineages and differentiation states of various human malignancies. In some embodiments, the methods described herein are used to quantify miRNA expression in different tissues, developmental lineages and/or different states of a condition. In some embodiments, the methods described herein are used to quantify miRNA expression in different states of a neoplastic and/or cancer condition.

In some embodiments, the method described herein are used to detect and/or quantify miRNA when less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the sample for further genetic analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for further genetic analysis.

In some embodiments, the methods described herein are used to detect and/or quantify target nucleic acids without the need of the specific target nucleic acid isolation. In some embodiments, the methods described herein are used to detect and/or quantify nucleic acids directly from a nucleic acid sample comprising DNA and RNA molecules.

In some embodiments, the methods described herein are used to detect and quantify nucleic acids in a subject originating from a different organism such as a virus or bacteria. In some embodiments, the methods described herein are used to detect viral nucleic acids. The methods described herein allow for discrimination and quantitation of different subtypes of virus. In some embodiments, the methods described herein can discriminate and quantitate different viral subtypes. The methods described herein can discriminate and quantitate at least 1, 2, 3, 4, 5, 10, 20, 50, 55, 60, 100, 500, 1000, 5,000, 10,000, 100,000 different viral subtypes. The methods described herein can discriminate and quantitate viral subtypes varying by 1 nt or more than 1, 2, 3, 4, 5, 10, 12, 15, 20 nt. In some embodiments, the methods described herein can discriminate and quantitate different viruses. The methods described herein can discriminate and quantitate at least 1, 2, 3, 4, 5, 10, 20, 50, 55, 60, 100, 500, 1000, 5,000, 10,000, 100,000 different viruses. The methods described herein can discriminate and quantitate viruses varying by 1 nt or more than 1, 2, 3, 4, 5, 10, 12, 15, 20 nt.

For instance, human papilloma virus (HPV) infections are associated with cervical cancers. Cervical cancer leads to about 250,000 deaths every year worldwide, majority in developing countries including China. Every year 470,000 new cases of cervical cancer are diagnosed. More than 70 types of HPV have been identified; some are the high-risk type associated with invasive cervical carcinoma and some are low-risk with benign proliferative diseases. HPV genotyping is critical to the early detection. In some embodiments, the methods described herein are used to detect and quantitate the presence of HPV in a sample. The methods described herein allow for discrimination and quantitation of the different subtypes of HPV. In some embodiments, the methods described herein can discriminate and quantitate the different HPV subtypes. The methods described herein can discriminate and quantitate at least 1, 2, 3, 4, 5, 10, 20, 50, 55, 60, 100 different HPV subtypes. The methods described herein can discriminate and quantitate HPV subtypes varying by 1 nt or more than 1, 2, 3, 4, 5, 10, 12, 15, 20 nt.

### Phage Promoter—OLA

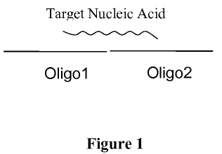

In one aspect, a set of oligonucleotides is designed to bind to a target nucleic acid. FIG. 1 shows an embodiment of the invention in which a pair of oligonucleotides (depicted as oligo 1 and 2 in FIG. 1) binds to a target nucleic acid. The methods described herein can be used in nucleic acid analysis including STR and SNP detection, RNA expression analysis, promoter methylation, gene expression, virus detection, viral subtyping and drug resistance.

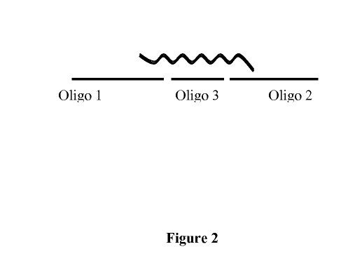

In some embodiments the set of oligonucleotide probes comprises a first oligonucleotide probe having a 5′ target specific region and a 3′ universal sequence region (depicted as oligo 1 in FIG. 1), and a second probe having a 3′ target specific region and a 5′ phage promoter region (depicted as oligo 2 in FIG. 1). In some embodiments, the first and the second oligonucleotide probes are suitable for ligation together when hybridized adjacent to one another to said target nucleic acid as shown in FIG. 1. In other embodiments, the oligonucleotide probe set comprises of a first oligonucleotide probe having a 5′ target specific region and a 3′ universal sequence region (depicted as oligo 1 in FIG. 2), a second probe having a 3′ target specific region and a 5′ phage promoter region (depicted as oligo 2 in FIG. 2) and a third probe having a target specific regions (depicted as oligo 3 in FIG. 2). The set of oligonucleotide probes can contain at least 2, 3, 4, 5, 6, 7, 8, 9, 10 oligonucleotide probes per target nucleic acid. For instance, the set of oligonucleotide probes can contain a first oligonucleotide probe having a 5′ target specific region and a 3′ universal sequence region, a second probe having a 3′ target specific region and a 5′ phage promoter region, a third probe having a target specific regions and a fourth probe having a target specific order. The four probes can be aligned when hybridized to the target nucleic acid in the following 3′ to 5′ order, first probe, third probe, fourth probe and second probe.

For simplicity, most of the examples and embodiments of the invention will be illustrated using a set of oligonucleotide probes containing a first and a second probe named oligo 1 and oligo 2 throughout the examples and embodiments described herein. However as mention above probe set containing more than two probes are encompassed in the methods described herein.

In some embodiments, the set of oligonucleotide probes comprises a first oligonucleotide probe having a 5′ target specific region and a 3′ universal sequence region (depicted as oligo 1 in FIG. 1). The universal region in oligo 1 can be the sequence of a promoter. In some embodiment, the promoter sequence in oligo 1 is a promoter for a DNA polymerase. Examples of DNA polymerase include, but are not limited to, Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase. In some embodiments, the promoter sequence in oligo 1 is a promoter for a phage polymerase. Examples of phage polymerase include, but are not limited to, T7 RNA polymerase, T3 RNA polymerase or SP6 RNA polymerase. In some embodiments, the universal sequence can be used to capture or detect the oligonucleotide set as described herein.

In some embodiments, the set of oligonucleotide probes comprises a second oligonucleotide probe having a 3′ target specific region and a 5′ phage promoter region (depicted as oligo 2 in FIG. 1). Examples of phage promoters include, but are not limited to, T7 RNA polymerase promoter, T3 RNA polymerase promoter or SP6 RNA polymerase promoter.

In some embodiments, the set of oligonucleotide probes comprises a first oligonucleotide probe having in a 3′ to 5′ order a universal sequence region, a tag region and a target specific region (depicted as oligo 1 in FIG. 1), where the universal region can be a promoter as described above. In some embodiments, the universal sequence can be used to capture or detect the oligonucleotide set as described herein. In some embodiments, the tag region of oligo 1 can be a unique sequence assigned to a specific target nucleic acid. The tag sequence can be used to capture or detect the oligonucleotide set as described herein.

In some embodiments, the set of oligonucleotide probes comprises a second oligonucleotide probe having in a 5′ to 3′ order a phage promoter region, a tag region and a target specific region (depicted as oligo 2 in FIG. 1), wherein the phage promoter region can be a promoter region as described above. In some embodiments, the tag region of oligo 2 can be a unique sequence assigned to a specific target nucleic acid. The tag sequence can be used to capture or detect the oligonucleotide set as described herein.

In some embodiments, the set of oligonucleotide probes binds to a target nucleic acid (as depicted as oligo 1 and 2 in FIG. 1). In some embodiments, either oligo 1 or oligo 2 have a capturing portion to separate the oligos bound to the target nucleic acid. The capturing portion can be a marker or a capturing sequence. In some embodiments, the capturing portion is a capturing sequencing. The capturing sequence can be the universal sequence of oligo 1 or the tag sequence of either oligo 1 or 2 as described above. The capturing sequence can be a new portion in oligo 1 or oligo 2 distinct from the universal sequence or tag sequences described above. In some embodiments, a capturing sequence is introduced at oligo 1, which can be captured by capturing sequence-conjugated to a solid structure such as beads or an oligonucleotide array. In some embodiments, a capturing sequence is introduced at oligo 2, which can be captured by capturing sequence-conjugated to a solid structure such as beads or an oligonucleotide array. In some embodiments, the capturing portion is a marker. Markers that are use to capture oligos are know in the art. The marker then can be captured by in a subsequent isolation step by a marker-binding solid structure. In some embodiments the marker is biotin. In some embodiments, biotin is introduced at oligo 1, which can be captured by streptavidin-conjugated to a solid structure such as beads. In some embodiments, biotin is introduced at oligo 2, which can be captured by streptavidin-conjugated to a solid structure such as beads. Biotin can be introduced at either oligo 1 or 2 by annealing a primer containing biotin to the universal sequence of oligo 1 or the phage promoter region of oligo 2. Alternatively, biotin can be introduced at either oligo 1 or 2 when the oligos are synthesized by methods known in the art.

In some embodiments, Oligo 1 will have a phosphate group at its 5′ end. Optionally, oligo 2 will have a T7 promoter at its 5′ end. When these two oligos simultaneously bind to one target nucleic acid molecule, e.g., mRNA, they are ligated according to techniques to known in the art. For example, the oligos can be ligated by T4 DNA ligase. When two oligos are stacking together to bind to a molecule with a perfect match at the junction, it results in a specific binding to the targeted nucleic acid, e.g., mRNA. The stacking oligos can be ligated to form one DNA molecule, which can be used for detection. Without being limited to any theory, any sequence-closely related to the target nucleic acid molecules will either block the ligation or prevent the hybrid formation. Therefore, isoforms can be distinguished in the assay. If the difference is in the middle of the target nucleic acid, it will block the ligation and detection, although the hybrids are able to form. In some embodiments multiple nucleic acids are analyzed by mixing multiple oligo sets together, each of which is specific to one nucleic acid target.

In some embodiments, either oligo 1 or oligo 2 have a capturing portion to separate the oligos as describe above. In some embodiments, after oligo 1 and 2 have been ligated the ligated product will be separated using the capturing portion in either oligo 1 or oligo 2. After separation, the ligated products of oligo 1 and oligo 2 can then detached from the duplexes and analyzed according to the methods described herein

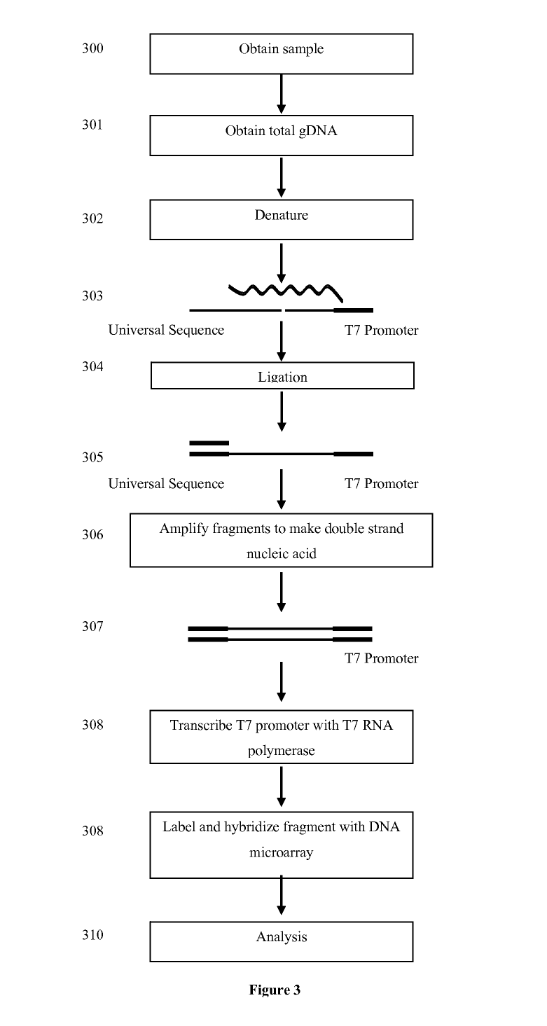

A quick overview for one of the embodiments of the invention is illustrated in FIG. 3. The methods described herein can be used in other nucleic acid analysis, including STR and SNP detection, RNA expression analysis, promoter methylation, gene expression, virus detection, viral subtyping and drug resistance.

First, in step 300 a sample is obtained from a subject such as a human according to standard methods known in the art. In step 301, nucleic acids (e.g. genomic DNA) is obtained from the sample. Nucleic acids are obtained from the sample using purification techniques known in the art. Generally, about 1 μg-2 μg of total nucleic acid is sufficient. Optionally, the target nucleic acid is analyzed in a mixture of total DNA and RNA. In step 302 the nucleic acid is denatured to allow the binding of oligo 1 and oligo 2. In step 303, oligo 1 and oligo 2 bind to the target nucleic acid. When the two oligos are stacking together to bind to a molecule with a perfect match at the junction, it results in a specific binding to the targeted nucleic acid. To analyze multiple nucleic acids, multiple oligo sets are mixed together, each of which is specific to one target nucleic acid. Each target nucleic acid molecule will initiate the formation of target nucleic acid/DNA duplex and multiple target nucleic acids lead to the assembly of multiple nucleic acid/DNA duplexes. The stacking oligos can be ligated to form one DNA molecule as depicted in step 304.

In some embodiments the 3′ end of the ligated probe product has capturing portion, e.g., biotin, attached thereto. The capturing portion can be used to separate the probes after hybridization with the target nucleic acid and/or ligation. The reaction mixture is contacted with a marker binding support, e.g., biotin binding support such as stretavidin bound to a solid support. This permits isolation of the target probe if no hybridization/ligation occurs or the isolating of hybridized/ligated probe product if hybridization/ligation has occurred. In some embodiments, the 3′ end of the ligated probe product has a marker, e.g., biotin, attached thereto and a label such as ³²P attached to the 5′ end. The reaction mixture is then contacted with a biotin binding support such as stretavidin bound to a solid support. This permits isolation of the target probe if no hybridization/ligation occurs or the isolating of the labeled hybridized/ligated probe product if hybridization/ligation has occurred.

In step 305 the ligated fragment having the universal sequence at the 3′ end is amplified using a primer that recognizes the universal sequence to form a double strand nucleic acid as depicted in step 306. The universal sequence can be the sequence of a promoter. The ligated fragment can be amplified using a DNA polymerase. Examples of DNA polymerases include, but are not limited to, Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase. The ligated fragment can be transcribed using a phage RNA polymerase. Examples of phage RNA polymerase include, but are not limited to, T7 RNA polymerase, T3 RNA polymerase or SP6 RNA polymerase. In some embodiments, the ligated DNA fragment serves as a template for in vitro transcription reaction. The in vitro transcription reaction is carried out in the presence of SP6 RNA polymerase.

In step 308, the double strand fragment having the T7 promoter sequence end is transcribed using T7 RNA polymerase. Optionally, the transcription reaction is carried out in the presence of a labeled nucleotide analog (e.g. biotin-CTP).

In any of the embodiments, amplification and/or transcription of ligated products may occur on a bead. In any of the embodiments herein, target nucleic acids may be obtained from a single cell.

In any of the embodiments herein, the nucleic acid(s) of interest can be pre-amplified prior to the hybridization or amplification step (e.g., PCR). In some cases, a nucleic acid sample may be pre-amplified to increase the overall abundance of genetic material to be analyzed (e.g., DNA). Pre-amplification can therefore include whole genome amplification such as multiple displacement amplification (MDA) or amplifications with outer primers in a nested PCR approach.

In steps 310 of FIG. 3, transcription product is analyzed. The transcription product can be labeled and hybridized with a DNA microarray (e.g., 100K Set Array or other array) according to standard methods known in the art. When the transcription reaction is carried out in the presence of a biotinylated nucleotide analog, the hybridized probes can be then detected, e.g., with HRP-conjugated streptavidin and a chemulinescent substrate.

In some embodiments, the methods described herein are used to detect and/or quantitate a nucleic acid from a virus. In some embodiments, the methods described herein are used to detect the presence of a virus in a sample. In some embodiments, the methods described herein are used to detect a virus subtype. Examples of virus that can be detected using the method described herein include, but are not limited to, human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses (HPV), orthomyxo viruses, paramyxo viruses, adenoviruses, corona viruses, rhabdo viruses, polio viruses, toga viruses, bunya viruses, arena viruses, rubella viruses, and reo viruses. In some embodiments, the methods described herein are used to detect a HPV subtype.

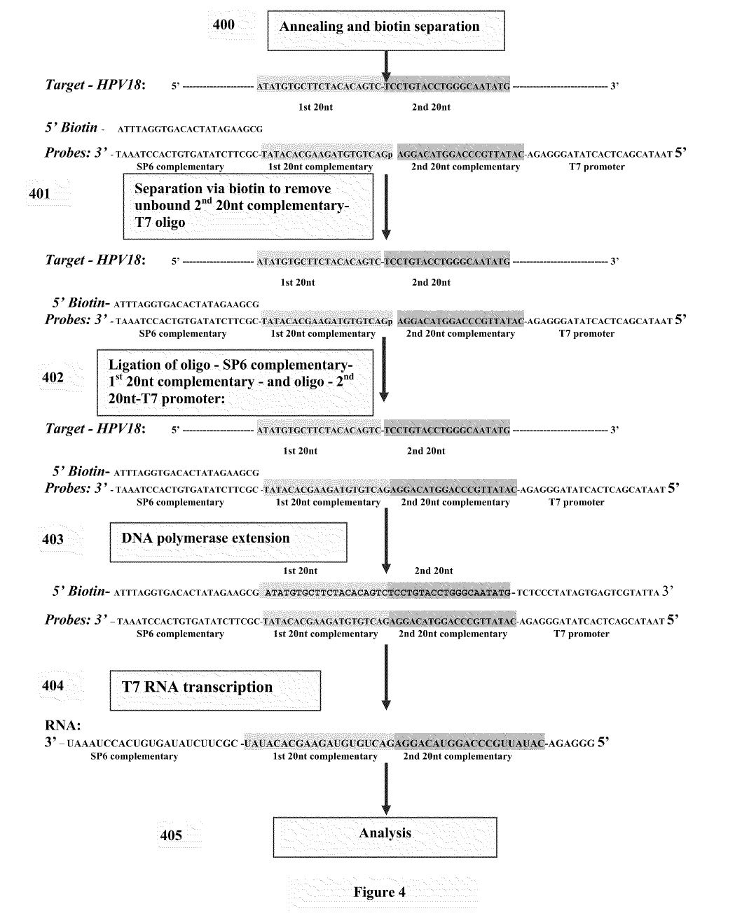

A quick overview for one of the embodiments of the invention to detect a HPV subtype is illustrated in FIG. 4. First, in step 400 the target HPV subtype, in this example HPV18 is allowed to binding of oligo 1 and oligo 2. When the two oligos are stacking together to bind to a molecule with a perfect match at the junction, it results in a specific binding to the targeted nucleic acid. The 3′ end of the ligated probe product has a biotin attached thereto via the binding of a primer containing biotin to the SP6 complementary region in the 3′ prime region of oligo 1. Biotin is used after hybridization to separate unbound oligo 2. The stacking oligos can be ligated to form one DNA molecule as depicted in step 402. In step 403 the ligated fragment having an SP6 complementary region at the 3′ end is amplified using a primer that recognizes the SP6 complementary to form a double strand nucleic acid as depicted in step 403. In step 404, the double strand fragment having the T7 promoter sequence end is transcribed using T7 RNA polymerase. Optionally, the transcription reaction is carried out in the presence of a labeled nucleotide analog (e.g. biotin-CTP). In step 405, the transcription product is detected and/or quantified using the methods described herein.

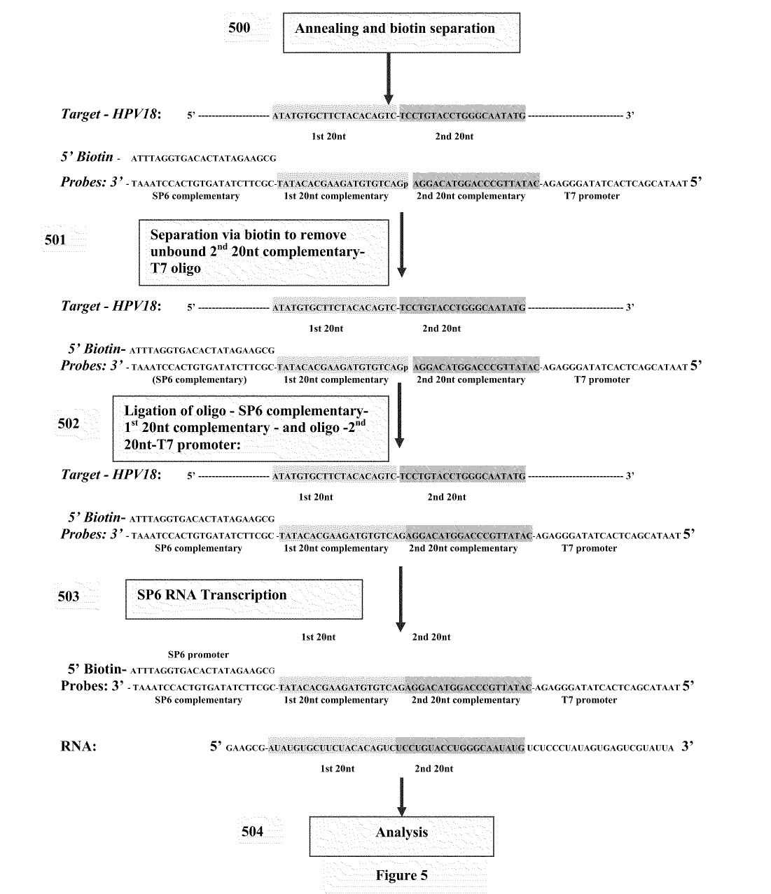

FIG. 5 shows a quick overview for another embodiment of the invention to detect a HPV subtype. First, in step 500 the target HPV subtype, in this example HPV18is allowed to binding of oligo 1 and oligo 2. When the two oligos are stacking together to bind to a molecule with a perfect match at the junction, it results in a specific binding to the targeted nucleic acid. The 3′ end of the ligated probe product has a biotin attached thereto via the binding of a primer containing biotin to the SP6 complementary region in the 3′ prime region of oligo 1. Biotin is used after hybridization to separate unbound oligo 2. The stacking oligos can be ligated to form one DNA molecule as depicted in step 402. In step 403 the ligated fragment having an SP6 complementary region at the 3′ end is transcribed using SP6 polymerase as depicted in step 403. Optionally, the transcription reaction is carried out in the presence of a labeled nucleotide analog (e.g. biotin-CTP). In step 404, the transcription product is detected and/or quantified using the methods described herein.

In some embodiments, the methods described herein are used to detect and/or quantitate a DNA epigenetic change. In some embodiments, the DNA epigenetic change is selected for the group consisting of chemical modifications and chromatin structure. In some embodiments the DNA epigenetic change is a chemical modification. In some embodiments, the chemical modification is DNA methylation. The present invention provides a method for determining methylation status of CpG dinucleotides within a target nucleic acid molecule. CpG islands (a stretch of CpGs), are typically unmethylated. Hypermethylation in CpG islands of promoter regions leads to silence the associated gene expression. Aberrant methylation has been associated to different pathogenesis including neoplasia. In some embodiments, to determine methylation, genomic DNA is treated with bisulfite. Unmethylated C is sensitive to the treatment of bisulfite, which converts to T, while methylated C is resistant to the modification. Therefore, determination of methylation and unmethylated C then becomes genotyping of C or T at a specific site. The methods described herein can be used then to genotype C or T at a specific site.

CpG islands are consisted of a stretch of CpGs, they are often close in the genome. Therefore, a pair of oligos used for a regular OLA analysis of a CpG site that is methylated or unmethylated is facing the problem of uncertainty with respect to the status of other CpG sites. In some embodiments, the methods of the invention use a degenerate oligonucleotides ligation assay (DOLA) that could be used for mapping methylation status of individual CpG sites within a bisulfite-treated genomic DNA. After bisulfite treatment, cytosine nucleotide of the target CpG site could be either C or T depending on methylation status of the CpG site. Two pairs of oligos are designed to cover these two possibilities, one for C and another for T. Because CpG sites are clustered within a CpG island and the methylation of these CpG sites is heterogeneous, the sequences for bisulfite converted genomic DNA is indecisive on the CpG sites. In order to ensure that the designed oligos match with the targeted DNA, degenerate oligos are designed to comprise all of possible methylation status. One of the degenerate oligos will anneal perfectly with the target sequence. After ligation, the methylation status of the target CpG site can be amplified or transcribed as described herein, e.g., by PCR or transcribed by T7 RNA polymerase, for further analysis consistent with the methods described herein. In some embodiment the methylation status of multiples CpG sites is profiled. A series of unique tag sequences to each CpG site can be incorporated in oligo 1 or oligo 2 as described above. A tag sequence then serves as a marker for a specific CpG site and can be detected in the analysis described herein, e.g., microarray analysis.

### Detection

In one aspect, at least one set of oligonucleotides probes is designed to bind to a target nucleic acid. The methods described herein can be used in nucleic acid analysis including STR and SNP detection, RNA expression analysis, promoter methylation, gene expression, virus detection, viral subtyping and drug resistance.

Results can be visualized by using a label in a microtiter plate. For instance, when the transcription reaction described in FIG. 3 is carried out in the presence of a biotinylated nucleotide analog, transcription product can be detected, e.g., with HRP-conjugated streptavidin and a chemulinescent substrate.

When analyzing target nucleic acids according to the methods described herein, the amplified/transcribed products of the ligated oligonucleotide probes can be labeled and hybridized with a DNA microarray (e.g., 100K Set Array or other array). Results can be visualized using a scanner that enables the viewing of intensity of data collected and software to detect and quantify nucleic acid. Such methods are disclosed in part U.S. Pat. No. 6,505,125. Another method contemplated by the present invention to detect and quantify nucleic acids involves the use of bead as is commercially available by Illumina, Inc. (San Diego) and as described in U.S. Pat. Nos. 7,035,740; 7033,754; 7,025,935, 6,998,274; 6,942,968; 6,913,884; 6,890,764; 6,890,741; 6,858,394; 6,812,005; 6,770,441; 6,620,584; G,544,732; 6,429,027; 6,396,995; 6,355,43 1 and US Publication Application Nos. 20060019258; 0050266432; 20050244870; 20050216207; 20050181394; 20050164246; 20040224353; 20040185482; 20030198573; 20030175773; 20030003490; 20020187515; and 20020177141; and in B. E. Stranger, et al., Public Library of Science-Genetics, I (6), December 2005; Jingli Cai, el al., Stem Cells, published online Nov. 17, 2005; C. M. Schwartz, et al., Stem Cells and Development, f 4, 517-534, 2005; Barnes, M., J. el al., Nucleic Acids Research, 33 (1 81, 5914-5923, October 2005; and Bibikova M, et al. Clinical Chemistry, Volume 50, No. 12, 2384-2386, December 2004. Additional description for preparing RNA for bead arrays is described in Kacharmina J E, et al., Methods Enzymol 303: 3-18, 1999; Pabon C, et al., Biotechniques 3 1(4): 8769,2001; Van Gelder R N, et a]., Proc Natl Acad Sci USA 87: 1663-7 (1990); and Murray, S S. BMC Genetics B(SupplI):SX5 (2005).

When analyzing SNP according to the methods described herein, the amplified/transcribed products of the ligated oligonucleotide probes can be labeled and hybridized with a DNA microarray (e.g., 100K Set Array or other array). Results can be visualized using a scanner that enables the viewing of intensity of data collected and software “calls” the SNP present at each of the positions analyzed. Computer implemented methods for determining genotype using data h m mapping arrays are disclosed, for example, in Liu, et al., Bioinformatics 19:2397-2403,2003; and Di et al., Bioinformatics 21: 1958-63,2005. Computer implemented methods for linkage analysis using mapping array data are disclosed, for example, in Ruschendorf and Nusnberg, Bioinfonnatics 21:2I23-5,2005; and Leykin et a]., BMC Genet. 6:7,2005; and in U.S. Pat. No. 5,733,729.

In some embodiments of this aspect, genotyping microarrays that are used to detect SNPs can be used in combination with molecular inversion probes (MIPS) as described in Hardenbol et al., Genome Res. 15(2):269-275,2005, Hardenbol, P. et al. Nature Biotechnology 2 1 (6), 673-8,2003; Faham M, et al. Hum Mol Genet. August 1; 10(16): 1657-64,200 1: Maneesh Jain, Ph.D., et aIl. Genetic Engineering News V24: No. 18,2004; and Fakhrai-Rad H, el aI. Genome Res. Jul; 14(7):1404-12, 2004; and in U.S. Pat. No. 5,858,412. Universal tag arrays and reagent kits for performing such locus specific genotyping using panels of custom MlPs are available from Affymetrix and ParAllele. MIP technology involves the use enzymological reactions that can score up to 10,000: 20,000, 50,000; 100,000; 200,000; 500,000; 1,000,000; 2,000,000 or 5,000,000 SNPs (target nucleic acids) in a single assay. The enzymological reactions are insensitive to crossreactivity among multiple probe molecules and there is no need for pre-amplification prior to hybridization of the probe with the genomic DNA. In any of the embodiments, the target nucleic acid(s) or SNPs can be obtained from a single cell.

Another method contemplated by the present invention to detect target nucleic acids involves the use of bead arrays (e.g., such as one commercially available by Illumina, Inc.) as described in U.S. Pat. Nos. 7,040,959; 7,035,740; 7033,754; 7,025,935, 6,998,274; 6,942,968; 6,913,884; 6,890,764; 6,890,741; 6,858,394; 6,846,460; 6,812,005; 6,770,441; 6,663,832; 5,520,584; 6,544,732; 6,429,027; 6,396,995; 6,355,431 m d US Publication Application Nos. 20060019258; 20050266432;20050244870;20050216207;20050181394;20050164246;20040224353:20040185482;200 30198573; 200301 75773; 20030003490; 200201 8751 5; and 200201 77 14 1; as well as Shen, R., et al. Mutation Research 573 70-82 (2005).

In any of the embodiments of this aspect, genotyping (e.g., SNP detection) and/or quantification analysis (e.g., RNA expression) of genetic content can be accomplished by sequencing. Sequencing can be accomplished through classic Sanger sequencing methods which are well known in the art. Sequence can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read. Sequencing can be preformed using genomic DNA, cDNA derived from RNA transcripts or RNA as a template.

In some embodiments of this aspect, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. This fast sequencing method also allows for detection of a SNP nucleotide in a sequence in substantially real time or real time. Finally, SMSS is powerful because, like the MIP technology, it does not require a pre amplification step prior to hybridization. In fact, SMSS does not require any amplification. SMSS is described in part in US Publication Application Nos. 2006002471 I; 20060024678; 20060012793; 20060012784; and 20050100932.

In some embodiments of this aspect, high-throughput sequencing involves the use of technology available by 454 Lifesciences, Inc. (Branford, Conn.) such as the Pico Titer Plate device which includes a fiber optic plate that transmits chemiluninescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density pricolitre reactors”, Nature, doi: 10.1038/nature03959; and well as in US Publication Apptication Nos. 200200 12930; 20030058629; 20030 1001 02; 20030 148344; 20040248 161; 200500795 10,20050 124022; and 20060078909.

In some embodiments of this aspect, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 200401061 30; 20030064398; 20030022207; and Constans, A, The Scientist 2003, 17(13):36.

In some embodiments of this aspect, high-throughput sequencing of RNA or DNA can take place using AnyDot.chjps (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO 02088382, WO 03020968, WO 0303 1947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science Feb. 16, 2001; Adams, M. et al, Science Mar. 24, 2000; and M. J, Levene, et al. Science 299:682-686, January 2003; as well as US Publication Application No. 20030044781 and 2006/0078937. Overall such system involve sequencing a target nucleic acid molecule having a plurality of bases by the temporal addition of bases via a polymerization reaction that is measured on a molecule of nucleic acid, i e., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable lo move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In any of the embodiment herein of this aspect, nucleic acids can be quantified. Methods for quantifying nucleic acids are known in the art and include, but are not limited to, gas chromatography, supercritical fluid chromatography, liquid chromatography (including partition chromatography, adsorption chromatography, ion exchange chromatography, size exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, and high-throughput genotyping such as with the use of molecular inversion probe (MIP).

Quantification of amplified target nucleic acid can be used to determine gene or allele copy number, gene or exon-level expression, RNA expression, methylation-state analysis, or detect a novel transcript in order to diagnose or condition, e.g. fetal abnormality, cancer or viral infection.

Detection and/or quantification of target nucleic acids can be done using fluorescent dyes known in the art. Fluorescent dyes may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine™), carboxy tetramethylrhodamine (TAMRA™), carboxy-X-rhodamine (ROX™), LIZ™, VIC™, NED™, PET™, SYBR, PicoGreen, RiboGreen, and the like. Descriptions of fluorophores and their use, can be found in, among other places, R. Haugland, Handbook of Fluorescent Probes and Research Products, 9.sup.th ed. (2002), Molecular Probes, Eugene, Oreg.; M. Schena, Microarray Analysis (2003), John Wiley & Sons, Hoboken, N. J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

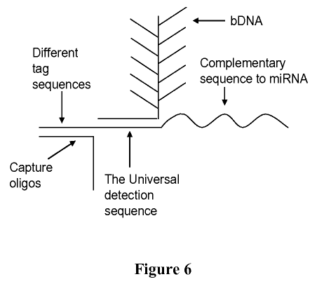

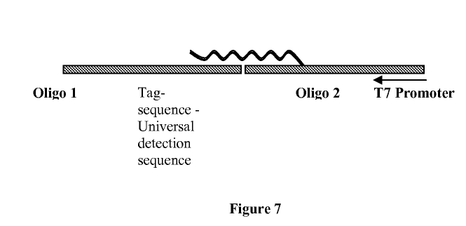

In another aspect of the invention, a branched-DNA (bDNA) approach is used to increase the detection sensitivity. In some embodiments, bDNA approach is applied to an array detection assay (FIG. 6). The array detection assay can be any array assay known in the art, including the array assays described herein. bDNA approach amplifies the signals through a branched DNA that are attached by tens or hundreds of alkaline phosphatase molecules. Thus, the signals are significantly amplified while the fidelity of the original nucleic acid target abundance is maintained. In some embodiments, a pair of oligonucleotides is designed to bind to a target nucleic acid (e.g., FIG. 1). In some embodiments, Oligo 1 will have a phosphate group at its 5′ end and a T7 promoter at its 5′ end. In some embodiments, a universal detection sequence is introduced in oligo 1. When these two oligos simultaneously bind to one target nucleic acid molecule, e.g., mRNA, they are ligated according to techniques to known in the art. For example, the oligos can be ligated by T4 DNA ligase. As no labeling, e.g., biotin labeling, is required in the detection using bDNA, in the embodiments where the ligated products are amplified and/or transcribed, amplification and/or transcription of the ligated product, e.g., oligo 1 and 2 can occur in the presence of regular NTPs. After hybridization via the tag sequence moieties, described herein, of the ligated products onto a substrate, (e.g. an array or beads), the universal detection sequence is then detected by bDNA (FIG. 6). Optionally, the amplified and/or transcribed product of the ligated oligos is hybridized onto a substrate (e.g. an array or beads). Because the signals are amplified, low abundant nucleic acids and nucleic acids in limited samples can be profiled. In some embodiments, a universal detection sequence is introduced through the tag sequences in oligo 1 (FIG. 7). In some embodiments, the ligation product of oligo 1 and 2 as described above is amplified and/or transcribed by any method known in the art including those described herein. After hybridization via the tag sequence moieties of the amplified and/or transcribed nucleic acids onto a substrate (e.g. beads or array), the universal detection sequence is then detected by bDNA. Because the signals are amplified, low abundant nucleic acids and nucleic acids in limited samples can be profiled.

### Heterogenous Annealing and Ligation

In one aspect of the invention, instead of performing the assay with soluble probes and thereafter immobilizing, one of the probes or the target nucleic acid may be immobilized on a solid support prior to annealing. In some embodiments, when one of these probes is immobilized, one of the other probes is labeled and in solution phase. This permits detection of label immobilized to the solid support based on the ligation. In some embodiments, when the target nucleic acid is immobilized, both the labeled and unlabeled probes are soluble in the fluid medium.

Techniques to immobilize nucleic acids, including the probes of the present invention, onto solid supports such as commercially available polymers, nylon, nitrocellulose membranes and dextran supports or beads are well known to those skilled in the art. Other immobilization techniques include attachment of biotinylated probes to immobilized streptavidin, the linking of amino groups on the probe to amino groups on a membrane bound protein support via a bifunctional linking reagent such as disuccinimidyl suberate and the methods described by Bischoff, et al. (1987), Anal. Biochem., 164, 336; Goldkorn, et al. (1986), Nucl. Acids Res., 14, 9171; Jablonski, et al. supra and Ghosh F., et al. (1987) Anal Biochem, 164, 336-344. Thus, for example, in one embodiment an adjacent probe may be bound to a solid support and contacted with a target nucleic acid under conditions which permit annealing of the adjacent probe to the complementary region of the target nucleic acid in a sample. Thereafter (or simultaneously therewith) the other probe(s) is contacted with the target nucleic acid to permit annealing of the target probe with the test DNA region immediately adjacent and contiguous to the adjacent probe. In some embodiments, one of the soluble probes contains a label. If necessary, the temperature is adjusted to maintain enzymatic activity of T4 DNA ligase which is thereafter contacted with the annealed target and adjacent probes to produce ligation if base pair matching in the end region of the target probe is present. Thereafter, the stringency of the fluid medium is raised to remove substantially all the species of the probes which are not ligated to the adjacent probe and/or target nucleic acid. The ligated product is then detected by standard techniques by measuring the ligated product bound to the solid support.

Alternatively, a biotinylated probe can be immobilized on a streptavidin-coated solid support (e.g., agarose beads).

The biotin-streptavidin binding phenomenon (or for that matter, any other binding phenomenon such as antibody-antigen binding, etc.) may also be utilized in a modified heterogenous assay. Thus, for example, one of the probes may be immobilized on a solid support by standard techniques. A biotinylated soluble probe is then employed in the assay as described. If ligation occurs the biotinylated ligated product will be bound to the solid support. Thereafter, any label linked to streptavidin, e.g., radioisotope, enzyme, etc. is contacted with the immobilized biotinylated linked probe product and assayed using standard techniques to ascertain whether the ligation event occurred.

It is also possible to assay for more than one target nucleic acid by using immobilized probes. Thus, sets of probes as described above each specific for one target nucleic acid may be employed. In some embodiments, each of the unlabeled probes from each probe set is immobilized in physically discrete sections on a solid support. In this manner, each discrete location represents a separate test for a particular target nucleic acid. Thereafter, the target nucleic acid is contacted with each of the immobilized probes. A mixture containing probes from each of the probe sets as described above is added. In some embodiment, a mixture containing labeled soluble probes from each of the above probe sets is then added. Each of these soluble probes is capable of annealing to the target nucleic acid and/or other probes in continuity with the immobilized probe. After ligation (if it occurs), non-ligated probes are removed from the solid support and ligated probe products immobilized on the solid support is detected. The detection of a ligated probe product in a particular discrete location on the support provides an indication of the presence or absence of the target nucleic acid.

Instead of immobilizing one of the probes, the target nucleic acid may also be immobilized to a solid support. Thus, for example, the target nucleic acid is transferred to, e.g., a nitrocellulose, nylon membrane or a bead by standard techniques.

### Kits

In an embodiment, a kit is provided for a detection and/or quantitation of a target nucleic acid. The kit includes: an oligo mix containing the oligonucleotide probes described herein. In addition, kits are provided which comprise reagents and instructions for performing methods of the present invention, or for performing tests or assays utilizing any of the compositions, arrays, or assemblies of articles of the present invention. The kits may further comprise buffers, restriction enzymes, adaptors, primers, a ligase, a polymerase, dNTPS, NTPs, detection reagents and instructions necessary for use of the kits, optionally including troubleshooting information.

### Methods

The methods described herein discriminate between nucleotide sequences. The difference between the target nucleotide sequences can be, for example, a single nucleic acid base difference, a nucleic acid deletion, a nucleic acid insertion, or rearrangement. Such sequence differences involving more than one base can also be detected. In some embodiments, the oligonucleotide probe sets have substantially the same length so that they hybridize to target nucleotide sequences at substantially similar hybridization conditions. As a result, the process of the present invention is able to detect infectious diseases, genetic diseases, and cancer. It is also useful in environmental monitoring, forensics, and food science. Examples of genetic analyses that can be performed on nucleic acids include e-g., SNP detection, STR detection, RNA expression analysis, promoter methylation, gene expression, virus detection, viral subtyping and drug resistance.

A wide variety of infectious diseases can be detected by the process of the present invention. Typically, these are caused by bacterial, viral, parasite, and fungal infectious agents. The resistance of various infectious agents to drugs can also be determined using the present invention.

Bacterial infectious agents which can be detected by the present invention include Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Fungal infectious agents which can be detected by the present invention include Cryptococcus neoformans, Blastomyces dermatitidis, Histoplasma capsulatum, Coccidioides immitis, Paracoccidioides brasiliensis, Candida albicans, Aspergillus fumigautus, Phycomycetes (Rhizopus), Sporothrix schenckii, Chromomycosis, and Maduromycosis.

Viral infectious agents which can be detected by the present invention include human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Parasitic agents which can be detected by the present invention include Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention is also useful for detection of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can all be identified with the present invention.

Genetic diseases can also be detected by the process of the present invention. This can be carried out by prenatal or post-natal screening for chromosomal and genetic aberrations or for genetic diseases. Examples of detectable genetic diseases include: 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Disease, thalassemia, Klinefelter Syndrome, Huntington Disease, autoimmune diseases, lipidosis, obesity defects, hemophilia, inborn errors of metabolism, and diabetes.

Cancers which can be detected by the process of the present invention generally involve oncogenes, tumor suppressor genes, or genes involved in DNA amplification, replication, recombination, or repair. Examples of these include: BRCA1 gene, p53 gene, APC gene, Her2/Neu amplification, Bcr/Ab1, K-ras gene, and human papillomavirus Types 16 and 18. Various aspects of the present invention can be used to identify amplifications, large deletions as well as point mutations and small deletions/insertions of the above genes in the following common human cancers: leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms.

In the area of environmental monitoring, the present invention can be used for detection, identification, and monitoring of pathogenic and indigenous microorganisms in natural and engineered ecosystems and microcosms such as in municipal waste water purification systems and water reservoirs or in polluted areas undergoing bioremediation. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can also be used in a variety of forensic areas, including for human identification for military personnel and criminal investigation, paternity testing and family relation analysis, HLA compatibility typing, and screening blood, sperm, or transplantation organs for contamination.

In the food and feed industry, the present invention has a wide variety of applications. For example, it can be used for identification and characterization of production organisms such as yeast for production of beer, wine, cheese, yogurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

### EXAMPLES

### Example 1

### Sample Preparation

Nucleic acids samples can be obtained from any tissue according to standard techniques known in the art.

a. miRNA Preparation

miRNA samples can be obtained from any tissue according to standard techniques known in the art. For instance, samples can be obtained from blood. For instance, miRNA samples can be obtained from white blood cells. Briefly, blood from a subject can be obtained in EDTA-containing blood collection tubes. Red blood cells are lysed by mixing the blood samples with 4 volumes of lysis buffer (10 mM Tris pH 8.0, 10 mM EDTA). After 10 min on ice with occasional agitation, the suspensions are centrifuged and the supernatants are decanted. The white blood cell pellets are resuspended in 20 ml of lysis buffer, and the above process is repeated. The white blood cells are then first lysed in a denaturing lysis solution which stabilizes RNA and inactivates RNases. The lysate is then extracted once with Acid Phenol:Chloroform which removes most of the other cellular components, leaving a semi-pure RNA sample.

Some of the methods describe herein do not need further purification of miRNA. However, in some embodiment a further isolation step may be performed. In order to perform this purification, the sample from above can be further purified according to standard techniques known in the art. For instance, the samples above can be further purified over a glass-fiber filter by one of two protocols from Ambion's mirVana™ miRNA isolation kit according to the manufacturer instructions to yield either total RNA or a size fraction enriched in miRNAs.

b. Genomic DNA Preparation

Genomic DNA samples can be obtained from any tissue according to standard techniques known in the art. For instance, samples can be obtained from blood. Genomic DNA can be prepared from the blood of subjects according to standard techniques known in the art. Briefly, blood can be obtained in EDTA-containing blood collection tubes. Red blood cells are lysed by mixing the blood samples with 4 volumes of lysis buffer (10 mM Tris pH 8.0, 10 mM EDTA). After 10 min on ice with occasional agitation, the suspensions are centrifuged and the supernatants are decanted. The white blood cell pellets are resuspended in 20 ml of lysis buffer, and the above process is repeated. Each cell pellet is then suspended in 15 ml of digestion buffer (50 mM Tris pH 8.0, 5 mM EDTA, 100 mM NaCl, 1% SDS) and 3 mg (0.2 mg/ml) of proteinase K is added. The cells are digested at 37° C. for 5 hours. The digests are extracted twice with equal volumes of phenol, then once with equal volumes of a 1:1 phenol:chloroform mixture and finally once with equal volumes of chloroform, each time centrifuging the mixture and removing the aqueous phase for the next extraction. After the final extraction and removing the aqueous phases, one tenth volume of 3 M sodium acetate, pH 6.5, is added. Two volumes of ice cold 100% EtOH are then added to each solution to precipitate the genomic DNAs, which are spooled out of solution on glass pipettes. The DNA precipitates are washed twice in 0.75 ml volumes of 70% EtOH, briefly centrifuging each time to allow removal of the supernatants. After removing the supernatants for the second time, the remaining EtOH is allowed to evaporate and the DNA is suspended in 0.5 ml of TE (10 mM Tri-HCl pH 8.0 containing 1 mM EDTA) solution. A fifth dilution of each DNA solution is also prepared in TE.

To determine the concentrations of the one fifth DNA solutions can be determined according to standard techniques known in the art.

To digest the genomic DNAs with Taq I, 25 μl of the 100 ng/μl solutions is mixed with 5 μl of 10× medium salt buffer (0.5 M NaCl, 0.1 M MgCl₂, 0.1 M Tris, pH 8.0), 20 μl of water-ME (i.e. water containing 6 mM ME (i.e., mercaptoethanol)), and 400 U of Taq I restriction endonuclease. The digests are covered with mineral oil and incubated at 65° C. for 1 hour. The reactions are stopped by adding 1.2 μl of 500 mM EDTA and heating the specimens to 85° C. for 10 min. Complete digestion of the DNAs is checked by electrophoresing aliquots on a 1% agarose gel.

### Example 2

### Oligonucleotide Preparation

Oligonucleotides can be synthesized according to standard techniques known in the art. For instance, oligonucleotides can be synthesized on a 394A DNA Synthesizer (Applied Biosystems Division of Perkin-Elmer Corp., Foster City, Calif.). Oligonucleotides labeled with Biotin can be synthesized using the manufacturer's suggested modifications to the synthesis cycle (Applied Biosystems Inc., 1994).

OLA oligonucleotides are purified by ethanol precipitation after overnight deprotection at 55° C. The primer-specific portions of the oligonucleotides used for PCR amplification are purified by polyacrylamide gel electrophoresis on 10% acrylamide/7M urea gels. Oligonucleotides are visualized after electrophoresis by UV shadowing against a lightening screen and excised from the gel (Applied Biosystems Inc., 1992). They are then eluted overnight at 64° C. in TNE (i.e. Tris-sodium EDTA) buffer (100 mM Tris/HCl pH 8.0 containing 500 mM NaCl and 5 mM EDTA) and recovered from the eluate using Sep Pak cartridges (Millipore Corp, Milford, Mass.) following the manufacture's instructions.

Oligonucleotides are resuspended in 100 μl TE (i.e. 10 mM Tri-HCl pH 8.0 containing 1 mM EDTA). Typical concentrations of these original OLA probe solutions are about 1 μg/μl or approximately 74 pm/μl.

As a prerequisite for the OLA phase, the downstream OLA oligonucleotides probes are phosphorylated with T4 polynucleotide kinase. Aliquots of the 5 downstream oligonucleotides equivalent to 200 pm are combined with 10 μl of 10× kinase buffer (500 mM Tris/HCl pH 8.0, 100 mM MgCl₂), 10 μl of 10 mM ATP, 20 U T4 kinase, and sufficient water-ME to give a final volume of 100 μl. Phosphorylation is carried out at 37° C. for 30 min followed by incubation for 10 min at 85° C. to inactivate the T4 enzyme.

The solutions of the OLA and PCR oligonucleotides are adjusted to convenient concentrations. The kinased OLA probe solution is diluted fourfold in water to yield a concentration of 1000 fm/μl. A solution of the upstream OLA probes is made by combining volumes of the probes equivalent to 200 pm with sufficient water to give a final volume of 400 μl. This created a solution 1000 fm/μl in each of the upstream OLA probes. Aliquots (20 μl) of the kinased and unkinased OLA probes are frozen for subsequent use.

Branched oligonucleotides can be synthesized according to any standard techniques known in the art. Branched oligonucleotides can be synthesized by chemical cross-linking of oligonucleotides containing three alkylamine functions as described in Clinical Chemistry (1993), 39(4): 725. Alternatively, branched oligonucleotides can be produced by incorporating “branching” monomers” during the chemical synthesis of oligodeoxyribonucleotides (Clinical Chemistry (1993), 39(4): 725). BMs are phosphoramidite reagents containing at least two protected hydroxyl functions. In general, a primary linear fragment is synthesized and then tailed with several appropriately spaced BMs. Several simultaneous secondary syntheses are then conducted from the branch points. Branched oligonucleotides containing several hundred nucleotides can be constructed in this way. Large-branched oligonucleotides for signal amplification can be synthesized by using a combination of solidphase chemistry and enzymatic ligation methods. For instance, an amplifier containing a maximum of 45 alkaline phosphatase probe-binding sites can be produced (1068 nucleotides). It can be constructed by synthesizing a bDNA with 15 branches (168 bases), which is then combined with a complementary linker that is in turn complementary to a branch extension (or “arm”; 60 bases), each of which has three binding sites for an alkaline phosphatase probe to bind (three sites times 15 branches=45 labels). The amplifiers are assembled by treatment with T4 DNA ligase, then analyzed by capillary electrophoresis.

FIG. 1 shows the design of OLA oligonucleotide probes for detection and quantification of miRNA in an OLA/PCR process. However, the oligonucleotides probes described herein can be use to determine any target nucleic acid of interested. In FIG. 1, these oligonucleotides are designed to specifically detect a single miRNA molecule. A pair of oligos is designed and synthesized, oligo 1 and oligo 2, to correspond to one miRNA molecule. Oligo 2 will have a phosphate group at its 5′ end. When these two oligos simultaneously bind to one miRNA molecule, they are ligated by T4 DNA ligase (FIG. 1). One of the oligos may non-specifically bind to a RNA or DNA molecule, but it would not result in detection, as these non-specific bindings of the oligos along with free oligos will be eliminated or removed by a separation as described below. When two oligos are stacking together to bind to a molecule with a perfect match at the junction, it results in a specific binding to the targeted miRNA. The stacking oligos can be ligated to form one DNA molecule, which can be used for detection. Any sequence-closely related miRNA molecules will either block the ligation or prevent the hybrid formation. Therefore, isoforms can be distinguished in the assay. If the difference is in the middle of the miRNA, it will block the ligation and detection, although the hybrids are able to form.

To analyze multiple miRNAs, e.g., in an array analysis, multiple oligo sets are mixed together, each of which is specific to one miRNA target. Each miRNA molecule will initiate the formation of RNA/DNA duplex and multiple miRNAs lead to the assembly of multiple RNA/DNA duplexes.

### Example 3

### T7-OLA Process

Materials: Oligo Mix (200 fmol/each target), Hybridization buffer, Streptavidin magnetic beads(Fisher), Beads washing buffer, ligase, ligation buffer (Femantas), Pre-reaction buffer, NTP mix(Roche), 10× T7 transcription buffer, T7 RNA transcriptase, Hybridization buffer, Hybridization washing solution, 1× Blocking buffer, Streptavidin-HRP conjugate, Washing buffer, Luminol/Enhancer Solution, Stable Peroxide Solution, Magentic stand (96 well plate or 24 well stand), PCR machine (for example. MJ), Hybridization oven, Washing tray, 0.2 ml or 0.4 ml tubes, Alpha Innotech image or equivalent image system or X-ray film

Hybridization of Target Nucleic Acid with Oligos:

a. Sample Preparation

From cultured cell lysate: Add 1 ml of cell lysate buffer per 1-2×10⁵ cells, and heat at 100° C. for 5 minutes and cool on ice, 40 μl is used for assay. From total RNA or DNA: Add 5 ul μl to 10 μl 1 ug-5 μg RNA or DNA, and heat at 72° C. for 5 minutes and cool on ice.

Incubate RNA or DNA sample with oligo mix through mixing the following components: 80 μl sample, 3 μl oligo mix, 15 μl hybridization buffer (500 mM NaCl, 20 mM Tris.HCl, 5 mMEDTA).

Incubate on PCR machine at 94° C. for 2 minutes, 55° C. for 10 minutes, and 35° C. for 1 hour.

Selection of Target Nucleic Acid/Oligo Hybrids:

a. Washing Beads

Add 5 μl beads with 150 μl of hybridization buffer in a tube, the size of the tube that should fit into the magnetic stand. Stay on the magnetic stand for 40 seconds. Aspirate out the liquid. Take out the tube from magnetic stand and add hybridization buffer, repeat one more time.

b. Beads Selection

Add 100 μl oligo mixture to the washed beads and resuspend the beads in solution. Incubate for 30 minutes. Put the bead mixture on the magnetic stand and stay for 30 second, and aspirate out the buffer. The beads remain on the side of tube. Remove the tube from the magnetic stand and add 150 μl of bead washing buffer (100 mM NaCl, 10 mMTris, Hcl, pH7.2, 5 mM EDTA, 0.1% Tween-20). Repeat the washing step for two times.

Ligation of target nucleic acid-directed pairing oligos to form a single molecule: The procedure is following to manufacturer's instruction. Add 50 μl of ligation buffer and put the tube on the magnetic stand for 30 seconds, remove the buffer. Add 1 μl ligase in 40 μl ligation buffer to make ligation mixture, completely resuspend the beads with ligation mixture. Incubate at room temperature for 1 hour.

Formation of double strand molecule: Add 80 ul bead washing buffer to ligated DNA, and put on the magnetic stand for 30 seconds, and remove the buffer, then add 20 ul reaction buffer (1.5 mM Mgcl2, 10 mM Tris-HCl, 50 mM KCL, 1 unit taq polymerase ), and incubate at 94° C. 30 seconds, 54° C. for 30 second, 72° C. for 45° C. to convert the single strand molecule to a double strand molecule. Using DNA polymerase and a primer specific to the universal region in oligo 1 the single strand molecule is converted into a double strand nucleic acid molecule.

T7 RNA transcription of ligated molecule: Put the reaction tube on the magnetic stand for 30 second. Transfer the 20 μl of reaction buffer to a fresh tube, and add 20 μl T7 RNA polymerase mixture containing: (i) 4 μl 5× T7 transcription buffer, (ii) 4 μl NTP mixture, (iii) 1 μl T7 RNA polymerase and (iv) 11 μl ddH₂O. Incubate at 37° C. for 1 hour. The reaction mixture is ready for further analysis.

### Example 4

### Bead Array Analysis

The arrays are spotted in triplicate, contain controls for monitoring hybridization specificity, include dye normalization controls, and have positive and negative controls spotted throughout the array.

The reaction mixture of Example 3 can be analyzed using arrays as described in Gunderson et al. Nature Genetics 37(5) 549-554, (2005). Oligonucleotide probes on the array are specific for the target nucleic acid, e.g. mRNA, and for the OLA probes. For, instance, the oligonucleotides can be 38 to 50 bases in length, ˜15 bases at the 5′ end and 3′ end for decoding and the remaining 20 bases are nucleic acid specific. The oligonucleotides are immobilized on activated beads using a 5′ amino group.

The reaction mixture is denatured at 95° C. for 5 min and then exposed to the Sentrix array matrix, which is mated to a microtiter plate, submerging the fiber bundles in 15 ml of hybridization sample. The entire assembly is incubated for 14-18 h at 48° C. with shaking. After hybridization, arrays are washed in 1× hybridization buffer and 20% formamide at 48° C. for 5 min.

For amplification and or transcription where biotin-dCTP is used, the biotin-labeled nucleotides are detected as described in Pinkel et al. PNAS 83 (1986) 2934-2938. The arrays are blocked at room temperature for 10 min in 1 mg ml⁻¹ bovine serum albumin in 1× hybridization buffer and then washed for 1 min in 1× hybridization buffer. The arrays are then stained with streptavidin-phycoerythrin solution (1× hybridization buffer, 3 μg ml⁻¹ streptavidin-phycoerythrin (Molecular Probes) and 1 mg ml⁻¹ bovine serum albumin) for 10 min at room temperature. The arrays are washed with 1× hybridization buffer for 1 min and then counterstained them with an antibody reagent (10 mg ml⁻¹ biotinylated antibody to streptavidin (Vector Labs) in 1× PBST (137 mM NaCl, 2.7 mM KCl, 4.3 mM sodium phosphate, 1.4 mM potassium phosphate and 0.1% Tween-20) supplemented with 6 mg ml⁻¹ goat normal serum) for 20 min. After counterstaining, the arrays are washed in 1× hybridization buffer and restained them with streptavidin-phycoerythrin solution for 10 min. The arrays are washed one final time in 1× hybridization buffer before imaging them in 1× hybridization buffer on a custom CCD-based BeadArray imaging system. The intensities are extracted intensities using custom image analysis software.

### Example 5

### Micro Array Analysis

Oligonucleotide probes on the array are specific for the target nucleic acid, e.g. mRNA, and for the OLA probes. For, instance, the oligonucleotides can be 38 to 50 bases in length, ˜15 bases at the 5′ end and 3′ end for decoding and the remaining 20 bases are nucleic acid specific. The oligonucleotides are immobilized on activated beads using a 5′ amino group. 5′ Amine oligonucleotides were resuspended in 1× Micro Spotting Plus buffer (ArrayIt, Sunnyvale, Calif.) at 20 μM concentration. Each oligonucleotide probe is printed four times on CodeLink-activated slides (GE health/Amersham Biosciences, Piscataway, N.J.) by a Pixsys7000 pin-based dispensing system (Genomics Solutions, Irvine, Calif.) in 2×2 pin and 40×8 spot configuration of each sub-array, with a spot diameter of 120 μm. The printed slides are further processed according to the manufacturer's recommendations. The array can also contains several 23 bp U6 and Drosophila tRNA oligonucleotides specifically designed as labeling and hybridization controls (positive) while 23 bp random oligonucleotides are designed as negative controls.

Hybridization buffer consists of 100 mM 2-(N-morpholino)ethanesulfonicacid (MES), 1 M [Na+], 20 mM EDTA, 0.01% Tween-20, and 0.5 mg/ml acetylated BSA. Target hybridization is done at 45° C. for 16 h, and slides are washed four times (6 min each) in buffer A (6× SSPE and 0.01% Tween-20) at RT, and then twice with buffer B (100 mM MES, 0.1 M [Na+] and 0.01% Tween-20) for 8 min at 45° C. Slides are then incubated for staining with Streptavidin solution mixture (100 mM MES, 1 M [Na+], 0.05% Tween-20, 2 mg/ml BSA and 10 μg/ml R-Phycoerythrin streptavidin) from Invitrogen at RT for 10 min followed by four washes with buffer A (6 min each) at 30° C.

Second staining is carried out with antibody solutions (100 mM MES, 1 M [Na+], 0.05% Tween-20, 2 mg/ml BSA, 0.1 mg/ml goat IgG and 5 μg/ml biotin anti-streptavidin) at RT for 10 min followed by washing with buffer A (twice) for 4 min. Third staining is performed with Streptavidin solution mixture at RT for 10 min and slides are washed four times (6 min each) with wash buffer A at 30° C. Finally, slides are washed one time, 5 min each at RT with 0.2×SSC and followed by a similar wash with 0.1×SSC to remove any salt remnant and binding particles to the slides.

### Example 6

### bDNA Analysis

Because a few of biotins are labeled on each probe and the templates for preparing probes are not amplified, the detection sensitivity is expected to be low and therefore this approach is not appropriate to profile those low abundant nucleic acid or nucleic acid in limited samples. To increase the detection sensitivity, a branched-DNA (bDNA) approach in the array detection (FIG. 6) can be used. Instead of template amplification like PCR, it amplifies the signals through a branched DNA that are attached by tens or hundreds of alkaline phosphatase molecules. Thus, the signals are significantly amplified while the fidelity of the original target nucleic acid abundance is maintained. First a universal detection sequence is introduced through extending the tag sequences in oligo 1 (FIG. 7). As no biotin labeling is required in the detection, transcription can take place in the presence of regular NTPs (see Example 3). After hybridization via the tag sequence moieties of the amplification products of the OLA reaction onto the array, the universal detection sequence is then detected by bDNA. Because the signals are amplified, low abundant nucleic acids, e.g., low abundant miRNAs and miRNA in limited samples, can be profiled.

The bDNA can then used in a solution-phase sandwich assay (see FIG. 6). The amplification products of the OLA reaction are denatured and hybridized in solution to two sets of oligonucleotide probes: the capturing probes with extensions and the labeling probes. Once the probe-target complex is bound to the well of the microtiter dish, the well is washed. The bDNA is then hybridized. After a wash, the bDNA is labeled with an alkaline phosphatase probe (18 bases). Finally, the complex is detected with a dioxetane substrate that can be triggered by an enzyme, (Lumigen, Detroit, Mich.) yielding a chemiluminescent output detectable with a luminometer.

bDNA assay procedure. Capture of the T7-OLA products on the microwell surface is accomplished by adding 200-μl aliquots of each T7-OLA product to the appropriate oligonucleotide-modified microwell. For the standard curve which is run on every assay plate, 50-μl aliquots of standards are added to the appropriate wells on the same microplate. The microplate then is sealed with high-density polyethylene sheets under silicon pads and incubated overnight (12 to 16 h) at 53° C. in a microwell plate heater (Chiron Corporation). The microwells are allowed to cool at room temperature for 10 min and then washed twice with wash A (0.13 SSC [13 SSC is 0.15 M sodium chloride plus 0.015 M sodium citrate], 0.1% sodium dodecyl sulfate). After incubation at 53° C. for 30 min with a 50-μl volume of preamplifier/amplifier diluent (prepare by incubating 50% horse serum, 1.3% sodium dodecyl sulfate, 6 mM Tris-HCl [pH 8.0], 53 SSC, and 0.5 mg of proteinase K per ml for 2 h at 65° C. and then adding 6 mM phenylmethylsulfonyl fluoride, 0.05% sodium azide, and 0.05% Proclin 300) containing 0.70 fmol of preamplifier (described above) per ml, the microwells are cooled and are washed as described above and then incubated at 53° C. for 30 min with 50 μl of preamplifier/amplifier diluent containing 1.0 fmol of bDNA amplifier per ml. After cooling and washing as described above, the microwells are incubated at 53° C. for 15 min with a 50-μl volume of label diluent (preamplifier/amplifier diluent plus 0.85% Brij 35, 0.85 mM ZnCₗ₂, and 17 mM MgCₗ₂) containing 0.40 fmol of label probe per ml. The microwells are cooled for 10 min and then are washed twice with wash A and twice with wash D (0.1 M Tris-HCl [pH 8.0], 2.5 mM MgCl₂, 0.1 mM ZnCl2, 0.1% Brij 35). A 50-μl volume of dioxetane substrate (Lumi-Phos Plus; Lumigen, Detroit, Mich.) is added to each microwell, and after incubation at 37° C. for 30 min, the luminescent output is measured by photon counting in a plate reading luminometer (Chiron Corporation).

The amount of amplification products of the OLA reaction in each specimen is quantified by using a standard curve. The assay standard can consist of a single-stranded DNA molecule. The single-stranded DNA standard is serially diluted in buffer to generate an eight-point standard curve. A calibration curve is generated from a least-squares quadratic polynomial fit in which the dependent variable was the log10 of the signal minus noise and the independent variable was the log10 of the amplification products of the OLA reaction quantification value assignment for each standard. Signal-minus-noise values for both the test samples and standards are calculated by subtracting the geometric mean relative luminescence of two wells containing only Base Matrix from the relative luminescence of each well containing either a sample or a standard.

T7-OLA product quantification values for each test sample are determined by calculating the mean log10 of the signal-minus-noise value, solving the quadratic equation for the log10 of the T7-OLA product quantification value, and then inverting back to the arithmetic scale. T7-OLA product quantification values are expressed in copies, where one copy is defined as the amount of T7-OLA product in a sample that generates a level of light emission equivalent to that generated by one copy of quality level 1 T7-OLA product reference material.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a target nucleic acid in a sample, comprising:
providing a sample potentially containing the target nucleic acid;
providing at least one oligonucleotide probe set, each set comprising a first oligonucleotide probe having a 5′ target specific region and a 3′ universal sequence region, and a second oligonucleotide probe having a 3′ target specific region and a 5′ phage promoter region, wherein the first and the second oligonucleotide probes are suitable for ligation together when hybridized adjacent to one another to said target nucleic acid;
annealing the oligonucleotide probe set to the target nucleic acid such that a complex is formed between the target nucleic acid and the oligonucleotide probes;
contacting the complex with a linking agent such that the directly adjacent 5′ and 3′ ends of the first and second probes covalently bond to form a ligated probe product;
annealing a primer to the 3′ universal sequence region of the first oligonucleotide probe in the ligated probe product;
contacting the annealed primer with a polymerase under conditions such that the annealed primer is extended to form an extension product complementary to the sequences to which the primer is annealed to form a double stranded nucleic acid product;
contacting the 5′ phage promoter region of the second oligonucleotide probe in the double stranded nucleic acid product with a phage polymerase under conditions such that a transcription product of said phage promoter region is formed; and
detecting the presence of the transcription product, wherein the presence of the transcription product is indicative of the presence of the target nucleic acid in the sample.

2. The method of claim 1, wherein said first and second oligonucleotide probes have a predetermined sequence.

3. The method of claim 1, wherein the phage promoter region of said second oligonucleotide probe is selected from the group consisting of T7 RNA polymerase promoter, T3 RNA polymerase promoter or SP6 RNA polymerase promoter.

4. The method of claim 1 wherein the universal sequence region of said first oligonucleotide probe is SP6 RNA polymerase promoter.

5. The method of claim 1, wherein said transcription product is detected using a DNA microarray, bead microarray, high throughput sequencing, or single microtiter plate assay.

6. The method of claim 1 wherein said transcription product is detected by binding a branched DNA to said transcription product.

7. The method of claim 1, wherein the transcription product has a detectable label.

8. The method of claim 7 wherein said detectable label is a fluorescent or biotin label, and the step of detecting includes detecting a fluorescent signal generated by the fluorescent or chemiluminescent or color.

9. The method of claim 7 wherein said label is incorporated during the transcription of said phage promoter region of said second oligonucleotide probe.

10. The method of claim 9 wherein said incorporation includes adding a label nucleotide to the transcription of said phage promoter region of said second oligonucleotide probe.

11. The method of claim 1 wherein said target nucleic acid is DNA or RNA.

12. The method of claim 11 wherein said DNA or RNA is derived from genomic DNA or total RNA.

13. The method of claim 1 further comprising separating the complex from the non-annealed first and second oligonucleotide probes.

14. The method of claim 1 wherein said first oligonucleotide probe further comprises a capturing portion.

15. The method of claim 14 wherein said capturing portion is used to separate the annealed complex from the non-annealed first and second oligonucleotide probes.

16. The method of claim 14 wherein said capturing portion is used to separate the ligated probe product from unligated first and second oligonucleotide probes.

17. The method of claim 14 wherein said capturing portion is biotin or a capture sequence.

18. The method of claim 17 wherein said capturing portion is biotin.

19. The method of claim 18 wherein said ligated probe product is isolated by binding said biotin with a strepavidin bound to a solid support.

20. The method of claim 1 wherein the primer annealed to the universal sequence of said first oligonucleotide further comprises a capturing portion.

21. The method of claim 20 wherein said capturing portion is used to separate the ligated probe product from unligated first and second oligonucleotide probes.

22. The method of claim 20 wherein said capturing portion is biotin or a capture sequence.

23. The method of claim 22 wherein said capturing portion is biotin.

24. The method of claim 23 wherein said ligated probe product is isolated by binding said biotin with a strepavidin bound to a solid support.

25. The method of claim 1 wherein said first oligonucleotide probe comprises in 5′ to 3′ order said target specific region, a tag region and said phage promoter region.

25. The method of claim 1 wherein said second oligonucleotide probe comprises in 3′ to 5′ order said target specific region, a tag region and said phage promoter region.
